(19) United States
(12) Patent Application Publication
(10) Pub. No.: US 2009/0149812 A1
(43) Pub. Date: Jun. 11, 2009

(54) NEEDLE GUIDANCE APPARATUS

(75) Inventor: Ewan MacAulay, South Australia (AU)

Correspondence Address: PEPPER HAMILTON LLP, ONE MELLON CENTER, 50TH FLOOR, 500 GRANT STREET, PITTSBURGH, PA, 15219, US

(73) Assignee: GRAMPIAN HEALTH BOARD, Aberdeen (GB)

(21) Appl. No.: 12/282,081

(22) PCT Filed: Mar. 6, 2007
(86) PCT No.: PCT/GB2007/000751

§ 371 Date: Jan. 9, 2009

(30) Foreign Application Priority Data
Mar. 6, 2006 (GB) 0604459.8

(51) Int. Cl. A61M 5/32

(52) U.S. Cl. 604/117

(57) ABSTRACT

The present invention relates to a needle guidance apparatus. The needle guidance apparatus comprises a needle guide configured for location in relation to a site on the human or animal body. The needle guidance apparatus is also configured to cooperate with a needle such that the needle is moveable at a predetermined angle in relation to the needle guide.

### FIELD OF THE INVENTION

The present invention relates to needle guidance apparatus suitable for providing access by a needle to a site on the human or animal body.

### BACKGROUND TO THE INVENTION

Vascular access is required for many medical procedures, such as haemodialysis, chemotherapy and other procedures involving intravenous medical administration and parenteral nutrition. In such medical procedures a fistula may be created in the body, e.g. on the underside of the forearm for haemodialysis, to provide a conduit that is capable of carrying a high blood flow. The fistula often needs to be capable of tolerating repeated needling. Alternatively, a vascular prosthetic graft may be surgically attached between an artery and a vein. Access to a fistula is normally gained by inserting two needles into the fistula at spaced apart locations. The inserted needles are then held in place whilst the medical procedure, e.g. dialysis, is performed. Likewise, a graft is brought into use by insertion of a needle into the graft. A rate of repetition of use of the fistula or graft depends on the medical procedure being performed. For example, dialysis is typically performed three times per week.

There are three methods of locating the spaced apart insertion points on a fistula. The three methods are the rotation method, the same area method and the exact-site method.

The rotation method has the advantage of allowing areas around the fistula to rest. However, the method has the disadvantage of a relatively high occurrence of bad sticks (i.e. where the needle misses the fistula), which increases the likelihood of complications, such as haematoma.

The same area method provides for ease of access to a fistula. However, use of the method often results in a weakening of the fistula wall and enlargement of the fistula in the area of the insertion points. This can increase the rate of complications, such as thrombosis, stenosis and false aneurysm formation, leading to loss of access to the fistula in addition to the attendant morbidity.

Use of the same site method provides for development of a track that can be accessed repeatedly. This technique also has been called the ‘constant-site’ method as described in Twardowski, Pol. Arch. Med. Wewn. 57:205-214, 1977 or the ‘exact-site’ or ‘buttonholing’ method. This method is preferred over the other methods because it is less likely to weaken the fistula wall, it is less painful, it reduces likelihood of haematoma formation, it increases the patency of the fistula, and it greatly reduces the need for reinsertion of needles as a result of ‘bad sticks’.

Of the above three methods the ‘exact-site’ method is preferred from a clinical perspective. However, it can be a difficult method to perform. More specifically, the fistula needs to be needled at the same site on at least six occasions to ensure that the track develops properly. Therefore, it is recommended that the same experienced member of staff should perform the procedure until the track develops properly to increase the likelihood that the needle is inserted repeatedly at the same site. As a result a patient is allocated a particular clinician or nurse to perform the procedure and this often presents difficulties because it is rare that the same member of staff will be available at each of the six required patient visits. In summary, there is a general reluctance to use the ‘exact-site’ method because of a lack of confidence on the part of staff and the complexity of managing duty rosters.

Where a graft is used, the angle of entry of a needle to the graft can have an impact on the complication rate. For example, if the angle of entry is too small the graft may be damaged by shearing; and if the angle of entry is too great there may be an increased likelihood of the back wall of the graft being punctured.

It is therefore an aim of the present invention to provide an apparatus that addresses the above noted problems. More specifically, it is an aim of the present invention to provide an apparatus that provides for access by a needle to site on the human or animal body without the need for experienced staff to perform the procedure.

### STATEMENT OF INVENTION

The present invention has been devised in the light of the above noted problems and thus according to a first aspect of the present invention there is provided a needle guidance apparatus comprising a needle guide configured for location in relation to a site on the human or animal body and being further configured to cooperate with a needle such that the needle is moveable at a predetermined angle in relation to the needle guide.

In use, the needle guide is located in relation to a site on the human or animal body, e.g. a vascular prosthetic graft or an arteriovenous fistula in a patient, and a needle brought into cooperation with and moved in relation to the needle guide so as to gain access to the site by the needle.

Configuring the needle guide such that the needle is movable in relation to the needle guide at a predetermined angle may, for example, provide for proper entry of a needle to a graft. For example, if insertion of a needle into a graft is attempted when an angle between the needle and the body is too shallow the needle may tear the graft. On the other hand, if the angle is too obtuse there can be an increased likelihood of the needle passing through the graft. Thus, configuring the needle guide such that a needle moves at a predetermined angle in relation to the needle guide can provide a means to set an appropriate angle of movement of a needle in relation to the body and, hence, the graft or fistula in the body.

More specifically, the needle guide may be configured such that the predetermined angle is between substantially 40 degrees and substantially 50 degrees.

More specifically, the needle guide may be configured such that the predetermined angle is substantially 45 degrees. An angle of 45 degrees has been found to be appropriate for gaining entry to a graft.

Alternatively or in addition, the needle guide may be configured such that the predetermined angle is between substantially 20 degrees and substantially 40 degrees.

More specifically, the needle guide may be configured such that the predetermined angle is substantially 30 degrees. An angle of 30 degrees has been found to be appropriate for gaining entry to a fistula.

Alternatively or in addition, the needle guide may have a needle guiding profile configured to cooperate with a needle and to guide movement of the needle in relation to the needle guide.

More specifically, the needle guiding profile may define a channel constructed to receive a needle. The channel may be constructed to restrict movement of the needle in relation to the needle guide radially of the channel.

More specifically, the channel may be substantially semi-circular in cross-section.

Alternatively or in addition, the channel may be substantially “U” shaped in cross-section. In use, the opposing of the channel may be spaced apart from each other to an extent that is greater than a width of a needle to reduce the likelihood of the needle slipping out of the needle guide.

Alternatively the channel may define a near complete circle in cross-section along at least part of its length. Thus the channel may have a gap which allows for removal of a needle from the channel when the needle has been inserted into the human or animal body where the needle is of a kind that tapers away from the inserted end of the needle.

Alternatively the channel may define a complete circle in cross-section along at least part of its length.

More specifically, the needle guide may comprise at least one channel closing component configured to be moved in relation to the channel from a first position in which the channel defines a complete circle along at least part of its length and to a second position in which the needle can be removed from the channel. Thus, in use, the channel closing component can be in the first position during movement of the needle in relation to the needle guide during insertion of the needle into the human or animal body and then can be moved to the second position to allow for removal of the needle from the channel of the needle guide when the needle is properly inserted into the human or animal body.

More specifically, the channel closing component may be frangible.

Alternatively or in addition, the channel closing component may be hingedly attached to the needle guide.

Alternatively or in addition, the needle guide may comprise a body engaging component configured to engage with the human or animal body.

More specifically, the body engaging component may be configured and the needle guiding profile disposed in relation to the body engaging component to provide for at least one predetermined attitude of the needle guiding profile in relation to the body engaging component. In use, this may provide for at least one predetermined attitude of the needle guiding profile in relation to the body.

More specifically, the needle guiding profile may be configured to provide for a plurality of predetermined attitudes of the needle guiding profile in relation to the body engaging component.

Alternatively or in addition, the needle guiding profile may be configured to receive needles of different gauges. Thus, the needle guiding profile may define a plurality of channels of different diameters.

Alternatively or in addition, the needle guide may comprise a plurality of channels of different diameters. The plurality of channels may be spaced apart from each other laterally of a direction of movement of a needle in a channel. Alternatively or in addition, the plurality of channels may be disposed substantially co-axially of each other.

Alternatively or in addition, the needle guide may be configured so as to permit alteration of the at least one predetermined attitude of the needle guiding profile in relation to the body. Thus, an angle between the needle guide and the body may be changed. For example, having an obtuse angle between the needle guide and the body initially may provide for ease of gaining access to the fistula by the needle; when the needle has entered the fistula the angle may be reduced so that the needle is substantially in line with the skin of the body.

More specifically, the needle guiding profile may be movable in relation to the body engaging component.

More specifically, the needle guiding profile may be rotatable in relation to the body engaging component.

More specifically, the needle guide may comprise a hinge configured to provide for rotation of the needle guiding profile in relation to the body engaging component.

Alternatively or in addition, the needle guide may be configured so as to permit stepwise alteration of the at least one predetermined attitude of the needle guiding profile in relation to the body.

Alternatively or in addition, the needle guide may be configured to resist an unintended reduction of a predetermined attitude of the needle guiding profile whilst permitting an intended increase in the predetermined attitude.

More specifically, the needle guide may comprise a ratchet arrangement.

More specifically, the ratchet arrangement may be configured to be releasable to provide for a reduction in the predetermined attitude of the needle guiding profile.

Alternatively or in addition, the body engaging component may be configured and the needle guiding profile may be disposed in relation to the body engaging component to provide for at least one predetermined limit to the extent to which a needle can be advanced through the needle guiding profile. Thus, in use, an extent to which the needle can be inserted into the human or animal body can be limited.

More specifically, the needle guiding profile may be configured to provide for a plurality of predetermined limits to the extent to which a needle can be advanced through the needle guiding profile and hence, in use, inserted into the human or animal body. For example, the needle guiding profile may be configured for use with a needle that is about 50 mm long, such as a 16 gauge needle. A 16 gauge needle is a typical arterial/venous fistula needle that is suitable for the majority of patients attending for kidney dialysis.

At a later stage, e.g. during the next patient visit, another needle is brought into cooperation with the needle guide apparatus so as to gain access to the said site. Thus, the needle guidance apparatus can be used to gain repeated access to the same site and thus provide for ease of use of the same site method.

Accordingly, the needle guide may be configured for temporary location on the human or animal body and may further comprise a registration profile for locating the needle guide in registration with the site on the human or animal body. This may provide for an improvement in the accuracy of the location of a point of insertion of a needle, whereby the risk of bad sticks may be reduced. Improved precision of location of the point of insertion may provide for repeated access to the same site as is required of the ‘exact site’ method. Thus, the needle guide can be removed from the human or animal body between uses of the needle guidance apparatus. Configuration of the needle guide for temporary location on the human or animal body and the provision of the registration profile can provide for removal of the needle guide from the body and replacement at a later stage whilst providing for repeated access to the site by a needle.

More specifically, the needle guide may comprise a registration component comprising said registration profile, the registration component being configured for cooperation with the human or animal body so as to provide for registration of the registration profile with a location on the human or animal body. For example, the registration profile may comprise an edge of the needle guide that is configured to rest on or near the skin of the human or animal body and that can be brought into registration with a mark made on the skin, such as a mark made by indelible marker pen.

Alternatively or in addition, the needle guidance apparatus may comprise a main body, which is configured to cooperate with the needle, and at least one registration component having a registration profile.

More specifically, the at least one registration component may extend from the main body.

More specifically, the needle guidance apparatus may comprise two registration components extending in opposite directions from the main body.

Where the needle guide comprises a body engaging component configured to engage with the human or animal body, the body engaging component may comprise a body engaging surface configured to be brought into contact with the skin of the human or animal body.

More specifically, the body engaging component may have a profile configured to permit positioning of the needle guide over a raised part of the skin of the human or animal body. For example, the raised part of the skin may be raised because of a fistula under the skin.

More specifically, the body engaging component may comprise an arcuate portion.

Alternatively or in addition, the body engaging component may be configured for use with differently shaped parts of the human or animal body.

Alternatively or in addition, the body engaging component may be pliable. Thus, the body engaging component may for example be more readily used with different parts of the human or animal body or with differently sized parts of the human or animal body. For example, where the body engaging component comprises a body engaging surface the body engaging surface can be shaped to conform to differently shaped forearms.

Alternatively or in addition, the body engaging component may be comprised at least in part of a plastics material.

Alternatively or in addition, the body engaging component may comprise hinged components configured to conform to differently shaped human or animal bodies. More specifically, a main body of the needle guide may be less pliable than the hinged components. Thus, the main body of the needle guide may provide for a needle to follow a fixed track as the needle advances into a fistula or graft.

Alternatively or in addition, the body engaging component may comprise at least in part a material that provides for friction between the body engaging component and the skin of the human or animal body. This can reduce the likelihood of the needle guide slipping on the human or animal body during use. More specifically, the body engaging component may comprise at least in part at least one of polypropylene (PP), polyphenylene sulphide (PPS) and polymethylpentane (PMP).

Alternatively or in addition, the body engaging component may comprise at least in part a material that provides for biocompatibility with the human or animal body. More specifically, the body engaging component may comprise at least in part at least one of polypropylene (PP), polyphenylene sulphide (PPS) and polymethylpentane (PMP).

Alternatively or in addition, the needle guide may be comprised at least in part of a plastics material.

Alternatively or in addition, the needle guide may be a unitary body.

The needle guidance apparatus may be configured for attachment to a tourniquet or similar such device.

In a form, the needle guidance apparatus may further comprise at least one needle configured to cooperate with the needle guide.

More specifically, the at least one needle may have a plurality of graduations spaced apart along the needle. The graduations may be spaced apart along a body of the needle. In use, the graduations can be used, e.g. by a clinician, to provide for determination of an extent to which the needle has been inserted into the human or animal body. Thus, the graduations may be discernible by a human.

More specifically, the plurality of graduations may comprise at least one of a coloured mark, engraved feature, a protrusion and other such visible feature.

Alternatively or in addition, the plurality of graduations may be spaced apart from one another by less than or equal to substantially 5 mm.

More specifically, the plurality of graduations may be spaced apart from one another by less than or equal to substantially 4 mm. Reduced spacing of the graduations may provide for improved resolution on the one hand but may be less readily perceived by the naked eye on the other hand.

More specifically, the plurality of graduations may be spaced apart from one another by less than or equal to substantially 3 mm.

Alternatively or in addition, the needle may comprise a bevelled aperture and at least one of the plurality of graduations may be disposed on at least one of a hilt of the needle and the needle itself so as to indicate an orientation of the bevelled aperture. A hilt of the needle may, for example, be formed of a plastics material.

Alternatively or in addition, the at least one needle may comprise an aperture (e.g. an eye) formed in a rear part of the needle. In use, the aperture may let part of the blood flow pass through the needle and thus, for example, through a graft during dialysis to thereby divert some of the blood flow into the dialysis unit.

Alternatively or in addition, the needle may comprise at least one member extending radially of the needle.

More specifically, the at least one member may comprise two members spaced apart circumferentially round the needle.

Alternatively or in addition, the at least one member may be formed of a plastics material.

Alternatively or in addition, the at least one member may be movable in relation to the needle.

More specifically, the at least one member may move circumferentially about the needle.

More specifically, the at least one member may be rotatable about the needle.

Alternatively or in addition, the at least one member may be attached to the needle such that the at least one member may be moved in relation to the needle. For example, the at least one member may be attached to the needle to provide for rotation of the at least one member about the needle.

In use, the at least one member may be held such that it extends away from the body as the needle is being moved in the needle guide. Thus, the at least one member can be used to provide for increased control of the needle, for example, as it is being inserted into a graft or fistula. Furthermore, the at least one member may be moved when the needle is in position in the body. For example, the at least one member may be moved to be in line with the skin of the body. In this disposition the at least one member may be used to hold the needle in position, e.g. by means of adhesive tape.

Alternatively or in addition, the at least one needle may be at least about 50 mm in length.

More specifically, the at least one needle may be about 60 mm in length.

Alternatively or in addition, the at least one needle may taper away from an inserted end of the needle.

Alternatively or in addition, a diameter of the at least one needle at a location spaced apart from an inserted end of the needle may be less than a diameter of the needle at a location towards the inserted end of the needle.

Alternatively or in addition, a first needle may have a tip configured to break the skin of the human or animal body. For example, the tip may be sharp. In use, the first needle can be used upon first use of the needle guidance apparatus when breaking the skin and perhaps also during formation of a fistula track, e.g. during the subsequent five, or more, uses of the needle guidance apparatus.

Alternatively or in addition, the first needle may have a surface configured to promote scarring of tissue at the site of insertion of the needle on the human or animal body. In use, this helps development of a fistula track suitable for subsequent repeated use.

Alternatively or in addition, at least a portion of the surface of the first needle may be rough. For example, a surface of the first needle at or towards an end configured to break the skin may be rough.

Alternatively or in addition, the needle guidance apparatus may further comprise a second needle configured to cooperate with the needle guide.

More specifically, the second needle may have a tip configured to reduce the likelihood of the tip breaking the skin of the human or animal body. For example, the tip of the second needle may be blunt. This can have the advantage of reducing the likelihood of pushing the needle through the back wall of the fistula, of needle-stick injury to the clinician, and of damaging the track between the skin and the fistula or the fistula itself.

Alternatively or in addition, the second needle may have a substantially smooth surface.

Alternatively or in addition, the second needle may have a diameter less than a diameter of the first needle. In use, this can provide for ease of insertion of the second needle into the human or animal body and can reduce damage to the fistula track.

Alternatively, the second needle may have diameter greater than a diameter of the first needle. In use, the second needle can provide for an increase in blood flow. Therefore, for example, more blood can be dialysed to thereby reduce the time required for dialysis treatment.

According to a second aspect of the present invention there is provided a kit of parts comprising: a needle guide configured for location in relation to a site on the human or animal body and being further configured to cooperate with a needle such that the needle is moveable at a predetermined angle in relation to the needle guide; and at least one needle.

More specifically, the kit of parts may comprise a first needle having a sharp tip, i.e. a tip configured to break the skin of the human or animal body.

Alternatively or in addition, the kit of parts may comprise a second needle having a blunt tip, i.e. a tip configured to reduce the likelihood of the tip breaking the skin of the human or animal body.

Further embodiments of the second aspect of the present invention may comprise one or more features of the first aspect of the present invention.

According to a third aspect of the present invention there is provided dialysis apparatus comprising needle guidance apparatus according to the first aspect of the present invention.

Embodiments of the third aspect of the present invention may comprise one or more features of the first aspect of the present invention.

According to a fourth aspect of the present invention, there is provided a needle configured to be inserted into the human or animal body, the needle comprising a body, a tip portion configured to be received in the body first upon insertion of the needle, and an aperture formed in the tip portion, in which a plurality of spaced apart graduations, which are discernible by a human, are disposed along a part of the body of the needle, the part of the body being spaced apart from the tip portion.

More specifically, further graduations may be disposed on the body of the needle between the part of the body bearing the plurality of graduations and the tip portion.

More specifically, the further graduations may be disposed on the tip portion.

Alternatively or in addition, the plurality of graduations may comprise at least one of a coloured mark, engraved feature, a protrusion and other such humanly discernible feature.

Alternatively or in addition, the plurality of graduations may be spaced apart from one another by less than or equal to substantially 5 mm.

Alternatively or in addition, the needle may be at least about 50 mm in length.

More specifically, the needle may be about 60 mm in length.

Alternatively or in addition, the needle may have a sharp tip portion, i.e. a tip portion configured to break the skin of the human or animal body.

Alternatively, the needle may have a blunt tip portion, i.e. a tip portion configured to reduce the likelihood of the tip portion breaking the skin of the human or animal body.

Alternatively or in addition, at least a part of a surface of the needle may be configured to promote scarring of tissue at the site of insertion of the needle on the human or animal body. In use, this helps development of a fistula track suitable for subsequent repeated use.

More specifically, at least a portion of the surface of the needle may be rough.

Alternatively, the needle may have a substantially smooth surface.

Further embodiments of the fourth aspect of the present invention may include at least one feature of the first to third aspects of the present invention.

According to a further aspect of the present invention there is provided a method of gaining access to a site on a human or animal body by a needle, the method comprising the steps of: locating a needle guide of a needle guidance apparatus in relation to a site on the human or animal body; bringing a needle into cooperation with the needle guide; and moving the needle at a predetermined angle in relation to the needle guide to gain access to the said site, in which the needle guide is configured for said method steps.

Embodiments of the further aspect of the present invention may comprise one or more features of the previous aspects of the present invention.

### BRIEF DESCRIPTION OF DRAWINGS

Further features and advantages of the present invention will become apparent from the following specific description, which is given by way of example only and with reference to the accompanying drawings, in which:

FIG. 1 is a perspective view of the needle guide of the present invention;

FIG. 2 is a plan view of the needle guide shown in FIG. 1;

FIG. 3 is an end view of the needle guide of FIG. 1;

FIG. 4 is a side view of the needle guide of FIG. 1;

FIGS. 5A to 5C are cross-sectional views through different embodiments of a channel of the needle guide of FIGS. 1 to 4;

FIG. 6 is a perspective view of a needle of the needle guidance apparatus of the present invention; and

FIG. 7 is a schematic of a hinge used in an embodiment of the needle guide of the present invention.

### SPECIFIC DESCRIPTION

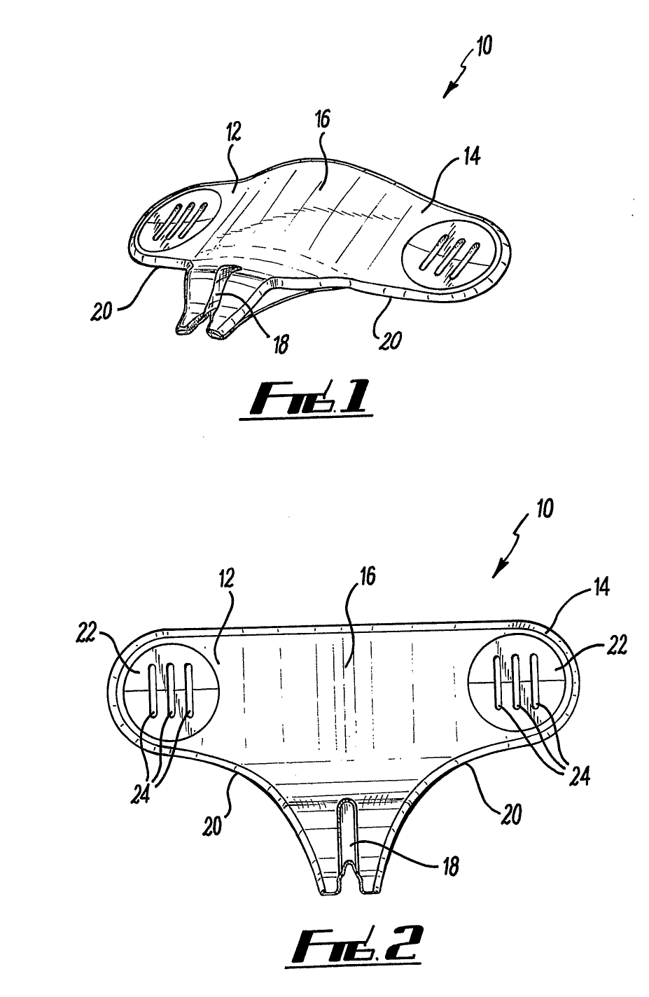

A needle guide 10 of needle guidance apparatus according to the present invention is shown in FIG. 1. The needle guide has two wing-shaped flaps 12, 14 extending laterally of a main body 16 that defines a channel 18 (which constitutes a needle guiding profile). The two flaps 12, 14 and main body 16 together constitute a body engaging component. The underside of the body engaging component (not shown in FIG. 1) defines a body engaging surface. Also, the two flaps 12, 14 constitute registration components and the leading edges 20 of the two flaps 12, 14 constitute registration profiles. As will become apparent from the description of use of the apparatus, which can be found below, other parts (e.g. the trailing edges) of the two flaps 12, 14 can constitute registration profiles.

The needle guide 10 is a unitary body made of a plastics material that provides for friction between the needle guide and the skin of a human or animal subject. Also, the plastics material is of a kind that is biocompatible with the skin of the human or animal subject.

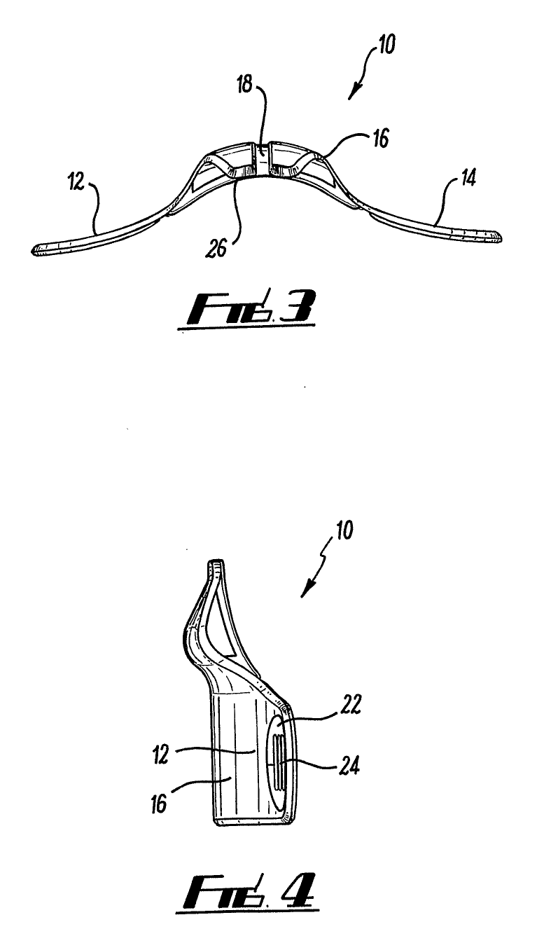

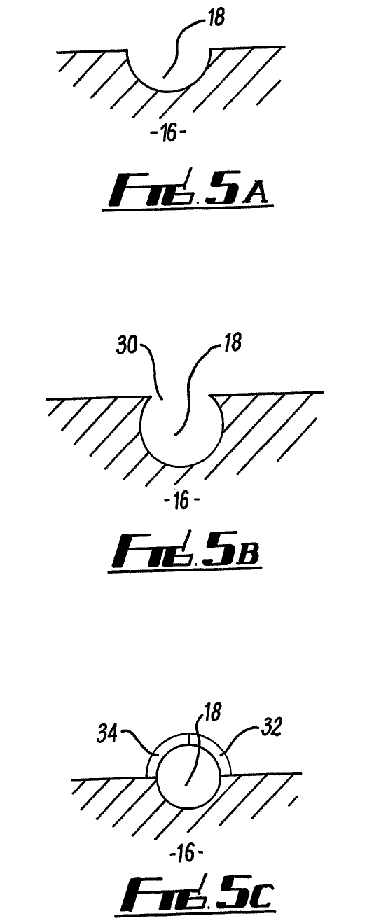

FIGS. 2 to 4 provide plan, end and side views of the needle guide 10 shown in FIG. 1. The needle guide of FIGS. 2 to 4 has components identified above with reference to FIG. 1 and thus the reader's attention is directed to the immediately preceding two paragraphs for a description of components common to FIG. 1 and to FIGS. 2 to 4. Components of FIGS. 2 and 3 not already described with reference to FIG. 1 will now be described with reference to each of FIGS. 2 and 3 in turn.

FIG. 2 shows the needle guide in plan view. A finger pad 22 is provided distally of each of the two flaps 12, 14. Each finger pad has ridges 24, which provide a grip for a finger of a user, e.g. the clinician, during use of the needle guide.

FIG. 3 shows an end view of the needle guide 10. As can be seen from FIG. 3 the two flaps 12, 14 and main body 16 comprise an arcuate portion 26. In use, the arcuate portion 26 provides for the spacing apart of the main body 16 from the skin of the human or animal subject. This can be useful where the needle guide 10 is being located over a raised part of the skin of the human or animal subject, such as a raised part created by a fistula located under the skin. The wing shaped flaps 12, 14 are pliable. This enables the needle guide to be used with differently shaped parts of a human or animal body, such as the forearm or leg, or indeed with differently sized parts of different human or animal bodies, such as forearms of different shape or size. In an un-illustrated embodiment, each of the two flaps 12, 14 is hinged in relation to the main body 16. The hinge may be formed by a conventional hinge mechanism or by means of a thin connecting member between each flap and the main body, with the thin connecting member being capable of repeated bending.

As can be seen from FIGS. 1 to 3, the main body 16 of the needle guide 10 defines a straight channel 18, which defines a predetermined angle in relation to the underside of the main body 16. Normally, the straight channel 18 defines an angle of 45 degrees, where access is to be gained to a graft, and 30 degrees, where access is to be gained to a fistula. Thus, the channel 18 is used to insert a needle at a 45 degree angle or a 30 degree angle, as appropriate, into the skin of the human or animal body. However, if a fistula is superficial or if the shape of the human or animal body so requires it, the straight channel 18 may define an angle of 25 degrees or less. In un-illustrated embodiments of the needle guide the main body 16 defines two or more channels, which are of different lengths, diameters or of different angles in relation to the underside of the main body. Channels of different lengths provide for different depths of penetration of the human or animal body by needles used in cooperation with the needle guide. Channels of different diameters provide for use of needles of different gauges in cooperation with the needle guide. Channels of different angles provide for the insertion of needles at different angles into the human or animal subject.

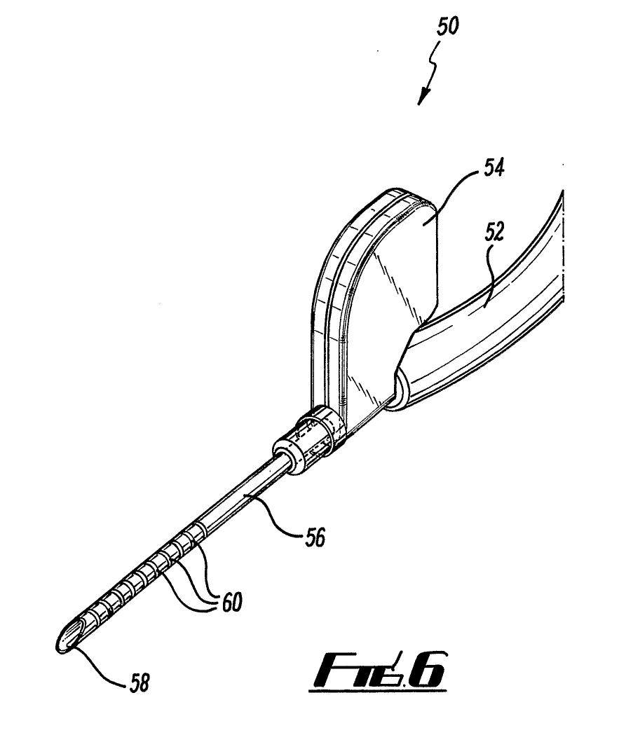

FIGS. 5A to 5C provide cross-sectional views through different embodiments of channel 18 of the needle guide of FIGS. 1 to 4.

As shown in FIG. 5A the channel 18 is of semi-circular cross-section.

In an alternative embodiment shown in FIG. 5B the channel 18 defines a near complete circle in cross-section to provide a gap 30. The gap 30 allows for removal of a needle from the channel 18 where the needle is of a kind having a diameter that is just less than the diameter of the channel towards the inserted end of the needle and less than the width of the gap away from the inserted end of needle. For example, the needle may be of a kind that tapers away from its inserted end.

In a further alternative embodiment shown in FIG. 5C the channel 18 defines a complete circle in cross-section. The channel is enclosed by frangible components 32, 34, which can be broken away from main body 16 to allow for removal of a needle from the channel 18. Alternatively hinged components 32, 34 can be provided instead of the frangible components. The hinged components 32, 34 hinge at the point where they join the main body 16 to provide for their re-use.

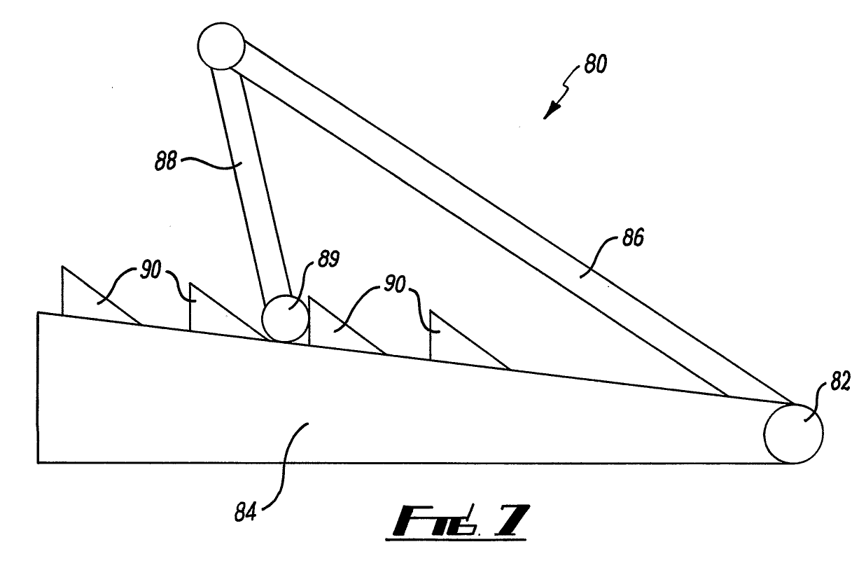

FIG. 6 provides a perspective view of a needle arrangement 50 of the needle guidance apparatus of the present invention. The needle arrangement 50 comprises a conduit 52, used for example during dialysis, a pair of flaps 54 and a needle 56. When the needle 56 has been properly inserted into the human or animal subject the flaps 54 are rotated apart from each other and used to hold the needle arrangement in place on the subject, e.g. by means of surgical tape bridging each flap 54 and the skin of the subject or similar such means of keeping the device in place. The needle 56 shown in FIG. 6 has a sharp tip 58, which is used to break the skin of the subject upon first use and during fistula development. The needle 56 also has a number of spaced apart ridges 60 towards the sharp tip 58. The ridges 60 provide a rough surface to the needle that promotes scarring of the tissue of the subject, which helps to develop formation of the fistula track. The ridges are also spaced apart from each other by a predetermined amount and are colour coded or incrementally numbered to provide visual feedback to the user, e.g. the clinician, of the extent to which the needle is inserted into the human or animal body.

When a fistula track has developed, a second (un-illustrated) needle is used. The second needle is the same as the needle shown in FIG. 6, with the exceptions that it has a blunt tip instead of a sharp tip 58 and a number of spaced apart coloured marks instead of ridges 60 such that the needle lacks a rough surface. Thus, the surface of the second needle is substantially smooth. A sharp tip and surface roughness are not needed on a needle after a fistula track has been developed. The second needle is of a smaller gauge than the first needle to provide for ease of insertion of the second needle into a developed fistula track.

The first 56 and second needles are about 50 mm in length.

Use of the needle guide and the needle arrangement will now be described with reference to FIGS. 1 to 6. The needle guide 10 is placed over the fistula present in the human or animal subject making use of the arcuate portion 26 where the fistula raises part of the skin of the subject. The pliability of the two flaps 12, 14 enables the user, e.g. a clinician, to conform the flaps to the shape of the part of the subject, e.g. forearm, bearing the fistula. Using an indelible marker pen reference marks are drawn on the skin of the subject using the leading edges 20 of the two flaps 12, 14 as a guide for the pen. The reference marks on the skin enable the needle guide to be removed and subsequently replaced on the subject in the same location ready for re-use. The needle 56 of a sharp tipped needle arrangement 50 is introduced into the upper end of the channel 18 of the needle guide 10 and the needle 50 moved through the channel until it breaks the skin. The needle is inserted into the subject to the required depth as indicated by the ridges 60 and the depth noted for subsequent use. The needle 56 is disengaged from the needle guide 10 as described above with reference to FIGS. 5A to 5C and the needle guide is removed from the subject. The needle 56 is then held in place on the subject by means of the flaps 54 as described above.

During subsequent uses of the needle guidance apparatus sufficient to develop the fistula track, the needle guide is located on the subject using the reference marks on the subject's skin and the needle arrangement 50 used as described in the immediately preceding paragraph. When the fistula track is properly developed the needle guidance apparatus can be used when required by making use of the needle guide 10 and the version of needle arrangement comprising the second needle, which has the blunt tip and the substantially smooth surface.

An embodiment of the present invention is shown in part in FIG. 7. The embodiment provides for movement of the channel 18 of the embodiment shown in FIGS. 1 to 3 in relation to the main body 16 of the embodiment of FIGS. 1 to 3. As shown in FIG. 7, the embodiment comprises a hinge arrangement 80 having a hinge 82 that provides for relative rotational movement of first and second parts 84, 86. The first part 84 forms part of the main body 16 and the second part 86 supports the channel 18. The hinge arrangement 80 also comprises an arm 88 that is rotatably connected at one end to the second part 86 distally of the hinge 82. The opposing free end 89 of the arm 88 is shaped to engage with a series of spaced apart teeth 90 that are mounted on the first part 84 so as to define an upwardly sloping stepwise progression of teeth. In use, the free end 89 engages with a particular tooth and sets the angle of the channel 18 with the main body 16. The teeth are shaped to resist an unintended reduction in the angle as might be caused by a clinician applying pressure accidentally to the channel 18 or the second part 86, which bears the channel 18. The angle between the channel 18 and the main body 16 can be increased by pushing the arm 88 such that the free end 89 engages with the next tooth in the upward slope. The angle between the channel 18 and the main body 16 can be deliberately reduced by rotating the second part 86 away from the first part 84 such that the free end 89 disengages from the teeth, whereby the first and second parts 84, 86 can then be moved together again whilst positioning the arm 88 such that it engages with a tooth 90 further down the slope defined by the teeth.

The embodiment of FIG. 7 enables the clinician to set an acute angle between the channel 18 and the main body 16 while the needle guide 10 is moved into position on a patient's body. The setting of an acute angle reduces the likelihood of a needle held in the channel 18 inadvertently breaking the patient's skin. When the needle guide 10 is in position the angle between the channel 18 and the main body can be increased as described above and as desired by the clinician before the needle is used to access a fistula under the patient's skin.

Aside from the above description of the first and second needles and their use with fistulae, needles having graduations disposed along their bodies have wider application as will now be described. The graduations are spaced from one another by about 5 mm. Four types of needle find use with grafts and fistulae. In all four types of needle the graduations are configured to indicate the orientation of the bevelled aperture of the needle. Also, all four types of needle have an eye in the back of the needle that provides for a flow of blood through the needle and thus through a fistula or graft, e.g. during dialysis. Furthermore, all four types of needle comprise a pair of flaps 54 as described above with reference to FIG. 6.

The first type of needle has a smooth surface, a sharp tip and is of a length of about 50 mm. This type of needle is used to access a graft using the above described needle guide 10 or to access deep grafts without the needle guide. More specifically, graduations are provided along the body of the needle as coloured marks such that they form no protrusions or concavities in the surface of the needle. The smooth surface minimises damage to the graft during needle entry. The sharp tip provides for a small size of puncture to the graft. In use, ultrasound is used to determine the depth of the graft below the surface of the skin. When the needle is inserted, the extent to which the needle is received in the body is monitored by means of the graduations vis-à-vis the depth of the graft to which access is being gained by the needle. If the needle has been advanced to the depth determined by ultrasound but the graft has not been penetrated, then there is a problem with the procedure and a risk presented of causing damage to the tissue. Thus, the needle should be withdrawn and the procedure repeated at another location.

The second type of needle has a rough surface, a sharp tip and is of a length of about 50 mm. The second type of needle is used along with the needle guide 10 to access fistulae and create buttonholes or without the needle guide to access deep fistulae. The graduations are etched into the surface of the needle such that they present a rough surface to the tissue upon insertion of the needle. The rough surface provided by the graduations and a lack of coating over the needle surface causes scarring of the tissue, which aids track development. The graduations enable the needle to be inserted to the same depth on each use. This aids development of the track along its entire length and reduces tapering of the track towards the fistula.

The third type of needle has a smooth surface, a blunt tip and is of a length of about 50 mm. This type of needle is used to access buttonholes using the needle guide 10 or to access deep fistulae without the needle guide. The blunt tip minimises damage to the track as the needle moves along the track to the fistula. The graduations provide the means to determine that the needle is inserted to the correct depth. The smooth surface of the needle and the formation of the graduations such that they form no protrusions or concavities in the surface of the needle minimises damage to the fistula during, needle entry.

The fourth type of needle has a smooth surface, a blunt tip and is of a length of about 25 mm. The fourth type of needle is used to access buttonholes without the needle guide 10. The fourth type of needle is as per the third type of needle, with the exception that the needle has a shorter length of about 25 mm.

What is claimed is:

1. A needle guidance apparatus comprising a needle guide configured to guide a needle to a site on a human or animal body, the needle guide comprising:
a body engaging component having a body engaging surface configured to be brought into contact with skin of the human or animal body; and
a needle guiding profile configured to cooperate with a needle to guide movement of the needle at a predetermined angle in relation to the needle guide.

2. The apparatus of claim 1 wherein the needle guiding profile defines an open channel configured to receive the needle and formed to restrict movement of the needle in relation to the needle guide radially of the channel.

3. The apparatus of claim 2 wherein the needle guide further comprises at least one channel closing component movable in relation to the channel from a first position to a second position, wherein in the first position the channel defines a complete circle along part of its length, wherein in the second position the needle is removable from the channel.

4. The apparatus of claim 3 wherein the channel closing component is at least one of:
frangible, and
hingedly attached to the needle guide.

5. The apparatus of claim 4 wherein the body engaging component is pliable.

6. The apparatus of claim 1, wherein the needle guide comprises a main body, which comprises comprising the needle guiding profile, and wherein the body engaging component comprises one or more hinged components extending from opposing sides of the main body, the one or more hinged components being pliable and hingedly movable in relation to the main body such that the one or more hinged components are conformable to differently shaped human or animal bodies.

7. The apparatus of claim 1, wherein the body engaging component has a profile configured to provide for positioning of the needle guide over a raised part of the skin of the human or animal body.

8. The apparatus of claim 7 wherein the profile is arcuate.

9. The apparatus of claim 1 wherein the needle guide is configured for temporary location on the skin of the human or animal body, the needle guide further comprising a registration profile configured for locating the needle guide in registration with the site on the skin of the human or animal body.

10. The apparatus of claim 9 wherein the needle guide further comprises two registration components extending in opposite directions from a main body of the needle guide, the main body comprising the needle guiding profile, and each registration component defining a registration profile.

11. The apparatus of claim 1, wherein the needle guiding profile is movable in relation to the body engaging component so as to provide for alteration of the predetermined angle of the needle guiding profile in relation to the body engaging component.

12. The apparatus of claim 11 wherein the needle guide is configured for stepwise alteration of the predetermined angle of the needle guiding profile in relation to the body engaging component.

13. The apparatus of claim 1 wherein the needle guide is configured such that the predetermined angle is between substantially 40 degrees and substantially 50 degrees.

14. The apparatus of claim 1 wherein the needle guide is configured such that the predetermined angle is between substantially 20 degrees and substantially 40 degrees.

15. A dialysis apparatus comprising a needle guidance apparatus, the needle guidance apparatus comprising a needle guide configured to guide a needle to a site on a human or animal body, the needle guide comprising:
a body engaging component having a body engaging component surface configured to be brought into contact with skin of the human or animal body; and
a needle guiding profile configured to cooperate with a needle to guide movement of the needle at a predetermined angle in relation to the needle guide.

16. The apparatus of claim 1, wherein the needle guide comprises a plurality of channels of different diameters for receiving needles of different gauges.

17. The apparatus of claim 16 wherein the channels are at least one of:
spaced apart from each other laterally of a direction of movement of a needle in a channel; and
disposed substantially co-axially of each other.

18. The apparatus of claim 11 wherein the needle guiding profile is rotatable in relation to the body engaging component.

19. The apparatus of claim 11 wherein the needle guide comprises a hinge operable to provide for rotation of the needle guiding profile in relation to the body engaging component.

20. The apparatus of claim 11 wherein the needle guide comprises a ratchet arrangement operable to resist an unintended reduction of the predetermined angle of the needle guiding profile whilst permitting an intended increase in the predetermined angle.

21-84. (canceled)
